(19) United States
(12) Patent Application Publication
(10) Pub. No.: US 2017/0251724 A1
(43) Pub. Date: Sep. 7, 2017

(54) FLEXIBLE DISPLAY FOR AN AEROSOL DELIVERY DEVICE

(71) Applicant: RAI Strategic Holdings, Inc., WINSTON-SALEM, NC (US)

(72) Inventors: Wilson Christopher Lamb, Hillsborough, NC (US); Frederic Philippe Ampolini, Winston-Salem, NC (US); Raymond Charles Henry, JR., Cary, NC (US)

(21) Appl. No.: 15/061,529

(22) Filed: Mar. 4, 2016

(51) Int. Cl. A24F 47/00; H05B 1/02; G08B 21/18; H05B 3/00

(52) U.S. Cl. CPC A24F 47/008; H05B 3/0014; H05B 1/0202; G08B 21/18

(57) ABSTRACT

An aerosol delivery device is provided that includes at least one housing equipped with a heating element and containing an aerosol precursor composition, the heating element being configured to activate and vaporize components of the aerosol precursor composition. The aerosol delivery device also includes a flexible, emissive or electronic paper display on and contoured to the at least one housing, and controllable to present information of the aerosol delivery device, such as an amount of remaining aerosol precursor composition or charge of a power source of the heating element.

### TECHNOLOGICAL FIELD

The present disclosure relates to aerosol delivery devices such as smoking articles, and more particularly to aerosol delivery devices that may utilize electrically generated heat for the production of aerosol (e.g., smoking articles commonly referred to as electronic cigarettes). The smoking articles may be configured to heat an aerosol precursor, which may incorporate materials that may be made or derived from, or otherwise incorporate tobacco, the precursor being capable of forming an inhalable substance for human consumption.

### BACKGROUND

Many smoking devices have been proposed through the years as improvements upon, or alternatives to, smoking products that require combusting tobacco for use. Many of those devices purportedly have been designed to provide the sensations associated with cigarette, cigar or pipe smoking, but without delivering considerable quantities of incomplete combustion and pyrolysis products that result from the burning of tobacco. To this end, there have been proposed numerous smoking products, flavor generators and medicinal inhalers that utilize electrical energy to vaporize or heat a volatile material, or attempt to provide the sensations of cigarette, cigar or pipe smoking without burning tobacco to a significant degree. See, for example, the various alternative smoking articles, aerosol delivery devices and heat generating sources set forth in the background art described in U.S. Pat. No. 7,726,320 to Robinson et al. and U.S. Pat. No. 8,881,737 to Collett et al., which are incorporated herein by reference. See also, for example, the various types of smoking articles, aerosol delivery devices and electrically-powered heat generating sources referenced by brand name and commercial source in U.S. Pat. Pub. No. 2015/0216232 to Bless et al., which is incorporated herein by reference. Additionally, various types of electrically powered aerosol and vapor delivery devices also have been proposed in U.S. Pat. Pub. Nos. 2014/0096781 to Sears et al. and 2014/0283859 to Minskoff et al., as well as U.S. patent application Ser. No. 14/282,768 to Sears et al., filed May 20, 2014; Ser. No. 14/286,552 to Brinkley et al., filed May 23, 2014; Ser. No. 14/327,776 to Ampolini et al., filed Jul. 10, 2014; and Ser. No. 14/465,167 to Worm et al., filed Aug. 21, 2014; all of which are incorporated herein by reference.

### BRIEF SUMMARY

The present disclosure relates to aerosol delivery devices, methods of forming such devices, and elements of such devices. In accordance with example implementations of the present disclosure, an aerosol delivery device may be equipped with a flexible, emissive or electronic paper display capable of present information such as an approximate remaining capacity of the device's power supply or aerosol precursor composition (e.g., in 10-20% increments), which may provide a user sufficient feedback to anticipate the need to charge or replace the power supply or cartridge. In some examples, the emissive or electronic paper display may be a flexible solution that does not include glass or other fragile components, and may be more easily contoured to various device form factors. The emissive or electronic paper display may be low-cost and moldable to various form factors of aerosol delivery devices. And the emissive or electronic paper display may be included on an aerosol delivery device or various of its components, such as its power supply or cartridge.

The present disclosure thus includes, without limitation, the following example implementations. In some example implementations, an apparatus comprises at least one housing equipped with a heating element and containing an aerosol precursor composition, the heating element being configured to activate and vaporize components of the aerosol precursor composition; and a flexible, emissive or electronic paper display on and contoured to the at least one housing, and controllable to present information of the aerosol delivery device, wherein the apparatus is an aerosol delivery device or a cartridge for an aerosol delivery device.

In some example implementations of the apparatus of the preceding or any subsequent example implementation, or any combination thereof, the emissive or electronic paper display is contoured to a non-planar surface of the at least one housing.

In some example implementations of the apparatus of any preceding or any subsequent example implementation, or any combination thereof, the non-planar surface is substantially tubular in shape.

In some example implementations of the apparatus of any preceding or any subsequent example implementation, or any combination thereof, the emissive or electronic paper display is controllable to present information that indicates an amount of aerosol precursor composition contained in or consumed from the at least one housing.

In some example implementations of the apparatus of any preceding or any subsequent example implementation, or any combination thereof, the emissive or electronic paper display being controllable to present information includes being controllable to present an icon that represents the aerosol precursor composition and indicates the amount thereof contained in or consumed from the at least one housing.

In some example implementations of the apparatus of any preceding or any subsequent example implementation, or any combination thereof, the heating element is connected or connectable with a power source, and the emissive or electronic paper display is controllable to present information that indicates an amount of charge contained in or consumed from the power source.

In some example implementations of the apparatus of any preceding or any subsequent example implementation, or any combination thereof, the emissive or electronic paper display being controllable to present information includes being controllable to present an icon that represents the power source and indicates the amount of charge contained in or consumed therefrom.

In some example implementations of the apparatus of any preceding or any subsequent example implementation, or any combination thereof, the emissive or electronic paper display being controllable to present information includes being controllable to present information that also indicates an amount of aerosol precursor composition contained in or consumed from the at least one housing.

In some example implementations of the apparatus of any preceding or any subsequent example implementation, or any combination thereof, the emissive or electronic paper display being controllable to present information includes being controllable to present an icon that represents the power source and indicates the amount of charge contained in or consumed therefrom, and an icon that represents the aerosol precursor composition and indicates the amount thereof contained in or consumed from the at least one housing.

In some example implementations, a control body is provided. The control body is coupled or coupleable with a cartridge that is equipped with a heating element and contains an aerosol precursor composition. In this regard, the control body is coupled or coupleable with the cartridge to form an aerosol delivery device in which the heating element is configured to activate and vaporize components of the aerosol precursor composition. The control body comprises at least one housing; a flexible, emissive or electronic paper display on and contoured to the at least one housing; and a control component contained within the at least one housing and configured to operate in an active mode in which the control body is coupled with the cartridge, the control component in the active mode being configured to control the heating element to activate and vaporize components of the aerosol precursor composition, the control component being further configured to control the emissive or electronic paper display to present information of the cartridge, control body or aerosol delivery device.

In some example implementations of the control body of any preceding or any subsequent example implementation, or any combination thereof, the emissive or electronic paper display is contoured to a non-planar surface of the at least one housing.

In some example implementations of the control body of any preceding or any subsequent example implementation, or any combination thereof, the non-planar surface is substantially tubular in shape.

In some example implementations of the control body of any preceding or any subsequent example implementation, or any combination thereof, the control component is configured to control the emissive or electronic paper display to present information that indicates an amount of aerosol precursor composition contained in or consumed from the at least one housing.

In some example implementations of the control body of any preceding or any subsequent example implementation, or any combination thereof, the control component being configured to control the emissive or electronic paper display includes being configured to control the emissive or electronic paper display to present an icon that represents the aerosol precursor composition and indicates the amount thereof contained in or consumed from the at least one housing.

In some example implementations of the control body of any preceding or any subsequent example implementation, or any combination thereof, the heating element is connected or connectable with a power source, and the control component is configured to control the emissive or electronic paper display to present information that indicates an amount of charge contained in or consumed from the power source.

In some example implementations of the control body of any preceding or any subsequent example implementation, or any combination thereof, the control component being configured to control the emissive or electronic paper display includes being configured to control the emissive or electronic paper display to present an icon that represents the power source and indicates the amount of charge contained in or consumed therefrom.

In some example implementations of the control body of any preceding or any subsequent example implementation, or any combination thereof, the control component being configured to control the emissive or electronic paper display includes being configured to control the emissive or electronic paper display to present information that also indicates an amount of aerosol precursor composition contained in or consumed from the at least one housing.

In some example implementations of the control body of any preceding or any subsequent example implementation, or any combination thereof, the control component being configured to control the emissive or electronic paper display includes being configured to control the emissive or electronic paper display to present an icon that represents the power source and indicates the amount of charge contained in or consumed therefrom, and an icon that represents the aerosol precursor composition and indicates the amount thereof contained in or consumed from the at least one housing.

These and other features, aspects, and advantages of the present disclosure will be apparent from a reading of the following detailed description together with the accompanying drawings, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as intended, namely to be combinable, unless the context of the disclosure clearly dictates otherwise.

It will therefore be appreciated that this Brief Summary is provided merely for purposes of summarizing some example implementations so as to provide a basic understanding of some aspects of the disclosure. Accordingly, it will be appreciated that the above described example implementations are merely examples and should not be construed to narrow the scope or spirit of the disclosure in any way. Other example implementations, aspects and advantages will become apparent from the following detailed description taken in conjunction with the accompanying drawings which illustrate, by way of example, the principles of some described example implementations.

### BRIEF DESCRIPTION OF THE FIGURES

Having thus described the disclosure in the foregoing general terms, reference will now be made to the accompanying drawings, which are not necessarily drawn to scale, and wherein:

FIG. 1 illustrates a perspective view of an aerosol delivery device including a cartridge, a control body and a flexible, emissive or electronic paper display, according to an example implementation of the present disclosure;

FIGS. 2 and 3 illustrate exploded views of respectively the cartridge and control body of FIG. 1, according to an example implementation of the present disclosure; and

FIG. 4 illustrates information that may be presented by the emissive or electronic paper display of FIG. 1, according to an example implementation of the present disclosure.

### DETAILED DESCRIPTION

The present disclosure will now be described more fully hereinafter with reference to example implementations thereof. These example implementations are described so that this disclosure will be thorough and complete, and will fully convey the scope of the disclosure to those skilled in the art. Indeed, the disclosure may be embodied in many different forms and should not be construed as limited to the implementations set forth herein; rather, these implementations are provided so that this disclosure will satisfy applicable legal requirements. As used in the specification and the appended claims, the singular forms “a,” “an,” “the” and the like include plural referents unless the context clearly dictates otherwise.

As described hereinafter, example implementations of the present disclosure relate to aerosol delivery systems. Aerosol delivery systems according to the present disclosure use electrical energy to heat a material (preferably without combusting the material to any significant degree) to form an inhalable substance; and components of such systems have the form of articles most preferably are sufficiently compact to be considered hand-held devices. That is, use of components of preferred aerosol delivery systems does not result in the production of smoke in the sense that aerosol results principally from by-products of combustion or pyrolysis of tobacco, but rather, use of those preferred systems results in the production of vapors resulting from volatilization or vaporization of certain components incorporated therein. In some example implementations, components of aerosol delivery systems may be characterized as electronic cigarettes, and those electronic cigarettes most preferably incorporate tobacco and/or components derived from tobacco, and hence deliver tobacco derived components in aerosol form.

Aerosol generating pieces of certain preferred aerosol delivery systems may provide many of the sensations (e.g., inhalation and exhalation rituals, types of tastes or flavors, organoleptic effects, physical feel, use rituals, visual cues such as those provided by visible aerosol, and the like) of smoking a cigarette, cigar or pipe that is employed by lighting and burning tobacco (and hence inhaling tobacco smoke), without any substantial degree of combustion of any component thereof. For example, the user of an aerosol generating piece of the present disclosure can hold and use that piece much like a smoker employs a traditional type of smoking article, draw on one end of that piece for inhalation of aerosol produced by that piece, take or draw puffs at selected intervals of time, and the like.

Aerosol delivery systems of the present disclosure also can be characterized as being vapor-producing articles or medicament delivery articles. Thus, such articles or devices can be adapted so as to provide one or more substances (e.g., flavors and/or pharmaceutical active ingredients) in an inhalable form or state. For example, inhalable substances can be substantially in the form of a vapor (i.e., a substance that is in the gas phase at a temperature lower than its critical point). Alternatively, inhalable substances can be in the form of an aerosol (i.e., a suspension of fine solid particles or liquid droplets in a gas). For purposes of simplicity, the term “aerosol” as used herein is meant to include vapors, gases and aerosols of a form or type suitable for human inhalation, whether or not visible, and whether or not of a form that might be considered to be smoke-like.

Aerosol delivery systems of the present disclosure generally include a number of components provided within an outer body or shell, which may be referred to as a housing. The overall design of the outer body or shell can vary, and the format or configuration of the outer body that can define the overall size and shape of the aerosol delivery device can vary. Typically, an elongated body resembling the shape of a cigarette or cigar can be a formed from a single, unitary housing or the elongated housing can be formed of two or more separable bodies. For example, an aerosol delivery device can comprise an elongated shell or body that can be substantially tubular in shape and, as such, resemble the shape of a conventional cigarette or cigar. In one example, all of the components of the aerosol delivery device are contained within one housing. Alternatively, an aerosol delivery device can comprise two or more housings that are joined and are separable. For example, an aerosol delivery device can possess at one end a control body comprising a housing containing one or more reusable components (e.g., an accumulator such as a rechargeable battery, thin film solid state battery and/or capacitor, and various electronics for controlling the operation of that article), and at the other end and removably coupleable thereto, an outer body or shell containing a disposable portion (e.g., a disposable flavor-containing cartridge).

Aerosol delivery systems of the present disclosure most preferably comprise some combination of a power source (i.e., an electrical power source), at least one control component (e.g., means for actuating, controlling, regulating and ceasing power for heat generation, such as by controlling electrical current flow the power source to other components of the article—e.g., a microprocessor, individually or as part of a microcontroller), a heater or heat generation member (e.g., an electrical resistance heating element or other component, which alone or in combination with one or more further elements may be commonly referred to as an “atomizer”), an aerosol precursor composition (e.g., commonly a liquid capable of yielding an aerosol upon application of sufficient heat, such as ingredients commonly referred to as “smoke juice,” “e-liquid” and “e-juice”), and a mouthend region or tip for allowing draw upon the aerosol delivery device for aerosol inhalation (e.g., a defined airflow path through the article such that aerosol generated can be withdrawn therefrom upon draw).

More specific formats, configurations and arrangements of components within the aerosol delivery systems of the present disclosure will be evident in light of the further disclosure provided hereinafter. Additionally, the selection and arrangement of various aerosol delivery system components can be appreciated upon consideration of the commercially available electronic aerosol delivery devices, such as those representative products referenced in background art section of the present disclosure.

In various examples, an aerosol delivery device can comprise a reservoir configured to retain the aerosol precursor composition. The reservoir particularly can be formed of a porous material (e.g., a fibrous material) and thus may be referred to as a porous substrate (e.g., a fibrous substrate).

A fibrous substrate useful as a reservoir in an aerosol delivery device can be a woven or nonwoven material formed of a plurality of fibers or filaments and can be formed of one or both of natural fibers and synthetic fibers. For example, a fibrous substrate may comprise a fiberglass material. In particular examples, a cellulose acetate material can be used. In other example implementations, a carbon material can be used. A reservoir may be substantially in the form of a container and may include a fibrous material included therein.

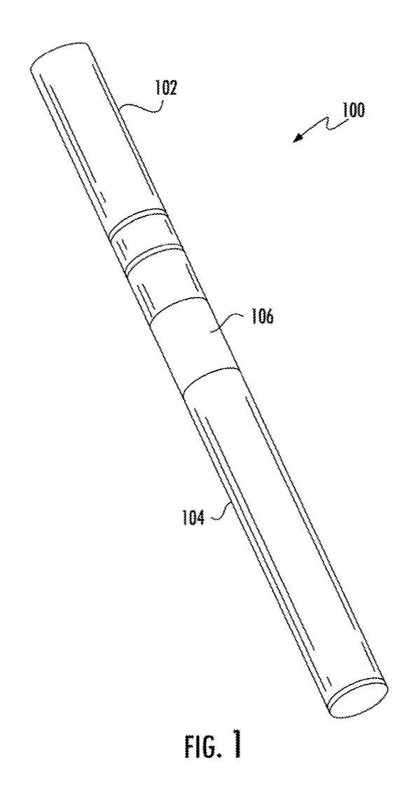

FIG. 1 illustrates a perspective view of an aerosol delivery device 100 including a cartridge 102 and a control body 104, according to various example implementations of the present disclosure. In particular, FIG. 1 illustrates the control body and the cartridge coupled to one another. The control body and the cartridge may be detachably aligned in a functioning relationship. Various mechanisms may connect the cartridge to the control body to result in a threaded engagement, a press-fit engagement, an interference fit, a magnetic engagement or the like.

The aerosol delivery device 100 may be substantially rod-like or tubular (or cylindrically) shaped in some example implementations when the cartridge 102 and the control body 104 are in an assembled configuration. In other examples, further shapes and dimensions are encompassed—e.g., a rectangular or triangular cross-section, multifaceted shapes, or the like. The aerosol delivery device may also be substantially rectangular or rhomboidal in cross-section, which may lend itself to greater compatibility with a substantially flat or thin-film power source, such as a power source including a flat battery. The cartridge and control body may include separate, respective housings or outer bodies, which may be formed of any of a number of different materials. The housing may be formed of any suitable, structurally-sound material. In some examples, the housing may be formed of a metal or alloy, such as stainless steel, aluminum or the like. Other suitable materials include various plastics (e.g., polycarbonate), metal-plating over plastic, ceramics and the like.

In some example implementations, one or both of the control body 104 or the cartridge 102 of the aerosol delivery device 100 may be referred to as being disposable or as being reusable. For example, the control body may have a replaceable battery or a rechargeable battery and thus may be combined with any type of recharging technology, including connection to a typical alternating current electrical outlet, connection to a car charger (i.e., a cigarette lighter receptacle), connection to a computer, such as through a universal serial bus (USB) cable or connector, or connection to a photovoltaic cell (sometimes referred to as a solar cell) or solar panel of solar cells. Further, in some example implementations, the cartridge may comprise a single-use cartridge, as disclosed in U.S. Pat. No. 8,910,639 to Chang et al., which is incorporated herein by reference in its entirety.

In some example implementations, the aerosol delivery device 100 may include a flexible, emissive or electronic paper display 106 on and contoured to the housing of either or both the cartridge 102 or control body 104 (shown), and controllable to present information of the aerosol delivery device. In some examples, a touch-sensitive surface may overlay the emissive or electronic paper display to form a touchscreen. In these examples, the touchscreen may be configured to both present information of the aerosol delivery device, and receive input to the aerosol delivery device.

In some examples, the emissive or electronic paper display 106 may be contoured to a non-planar surface of the housing of the cartridge 104 or control body 104, such as a surface that is substantially tubular in shape. In the emissive or electronic paper display may be controllable by the control component to present information of the control body, cartridge or aerosol delivery device 100. Examples of suitable emissive displays include those based on technologies such as amorphous silicon (a-Si), low-temperature a-Si, low-temperature polycrystalline silicon (LTPS), organic semiconductor processes, jet-printed electronics and the like. More specific examples of suitable emissive displays include a-Si thin-film transistor (TFT) displays, LTPS organic light emitting diode (OLED) displays, active matrix OLED (AMOLED) displays, and the like. Examples of suitable electronic paper displays include electronic paper or e-paper displays such as Gyricon displays, electrophoretic displays, microencapsulated electrophoretic displays, electro-wetting displays, electrofluidic displays, interferometric modulator displays, other bistable displays and the like.

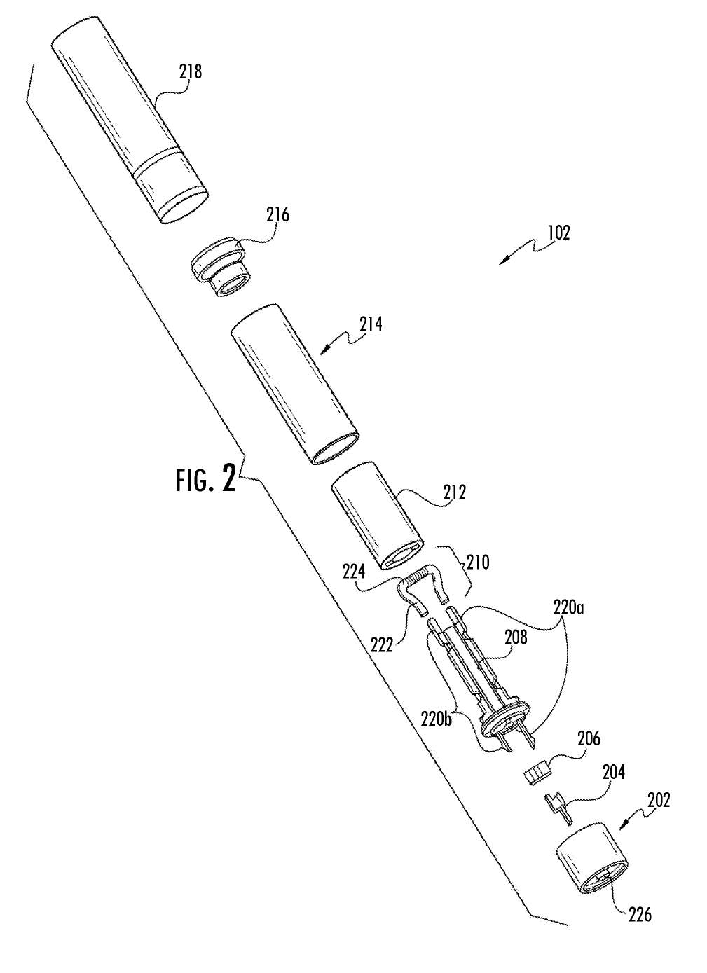

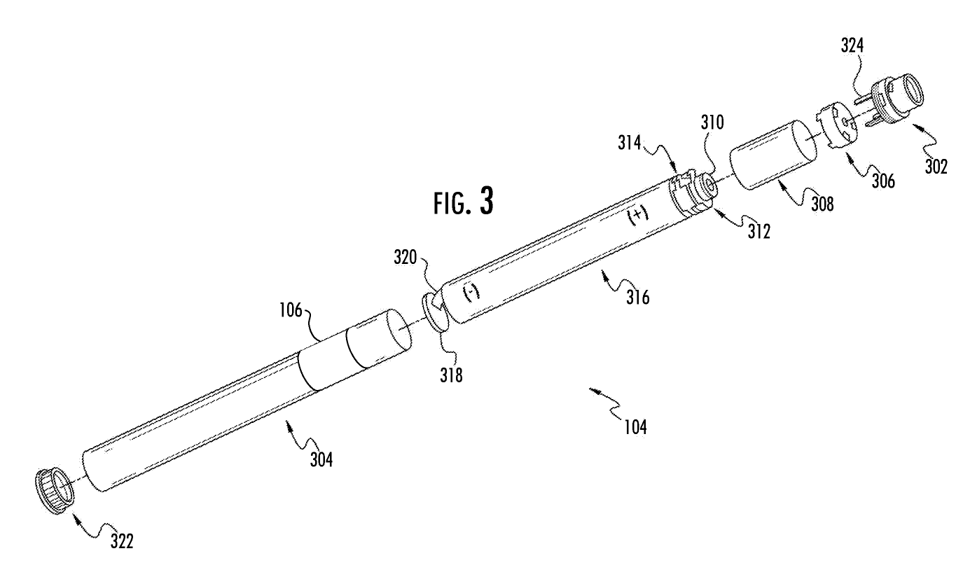

FIGS. 2 and 3 illustrate exploded views of respectively the cartridge 102 and control body 104 of FIG. 1, according to an example implementation of the present disclosure in which the control body may include an emissive or electronic paper display 106. The components illustrated in FIGS. 2 and 3 are representative of the components that may be present in a cartridge and control body and are not intended to limit the scope of components that are encompassed by the present disclosure. It should also be understood that in other example implementations, the cartridge may include an emissive or electronic paper display in addition to or in lieu of the control body.

As illustrated in FIG. 2, the cartridge 102 may include a base 202, a control component terminal 204, a (electronic) control component 206, a flow director 208, an atomizer 210, a reservoir substrate 212, an outer body 214, a mouthpiece 216, a label 218, and first and second heating terminals 220a, 220b. The atomizer may include a liquid transport element 222 and a heating element 224 (sometimes referred to more simply as a heater). In some examples, the cartridge may additionally include a base shipping plug engaged with the base and/or a mouthpiece shipping plug engaged with the mouthpiece in order to protect the base and the mouthpiece and prevent entry of contaminants therein prior to use as disclosed, for example, in U.S. Pat. App. Pub. No. 2014/0261408 to Depiano et al. In various configurations, this structure may be referred to as a tank; and accordingly, the terms “cartridge,” “tank” and the like may be used interchangeably to refer to an outer body or other housing enclosing a reservoir (reservoir substrate) for aerosol precursor composition, and including a heating element.

The base 202 and mouthpiece 216 may be coupled to respectively first and second opposing ends of the outer body 214 to enclose the remaining components of the cartridge 102 therein. The base may be configured to engage the control body 104. In some examples, the base may include anti-rotation features that substantially prevent relative rotation between the cartridge and the control body as disclosed in U.S. Pat. App. Pub. No. 2014/0261495 to Novak et al., which is incorporated herein by reference in its entirety. The label 218 may at least partially surround one or more of the outer body, base or mouthpiece, and include information such as a product identifier thereon.

Various components may be received within the outer body 214 and positioned between the base 202 and the mouthpiece 216. For example, the control component terminal 204, the control component 206, the flow director 208, the atomizer 210, and the reservoir substrate 212 may be retained within the outer body. The atomizer may include a first heating terminal 220a and a second heating terminal 220b, a liquid transport element 222 and a heating element 224. In this regard, the reservoir substrate may be configured to hold an aerosol precursor composition, which is directed to the heating element via the liquid transport element, as described below.

The aerosol precursor composition, also referred to as a vapor precursor composition, may comprise a variety of components including, by way of example, a polyhydric alcohol (e.g., glycerin, propylene glycol or a mixture thereof), nicotine, tobacco, tobacco extract and/or flavorants. Representative types of aerosol precursor components and formulations also are set forth and characterized in U.S. Pat. No. 7,217,320 to Robinson et al. and U.S. Pat. Pub. Nos. 2013/0008457 to Zheng et al.; 2013/0213417 to Chong et al.; 2014/0060554 to Collett et al.; 2015/0020823 to Lipowicz et al.; and 2015/0020830 to Koller, as well as WO 2014/182736 to Bowen et al, the disclosures of which are incorporated herein by reference. Other aerosol precursors that may be employed include the aerosol precursors that have been incorporated in the VUSE® product by R. J. Reynolds Vapor Company, the BLU™ product by Imperial Tobacco Group PLC, the MISTIC MENTHOL product by Mistic Ecigs, and the VYPE product by CN Creative Ltd. Also desirable are the so-called “smoke juices” for electronic cigarettes that have been available from Johnson Creek Enterprises LLC.

The reservoir substrate 212 may include a plurality of layers of nonwoven fibers formed into the shape of a tube encircling the interior of the outer body 214 of the cartridge 102. Liquid components, for example, can be sorptively retained by the reservoir substrate. The reservoir substrate is in fluid connection with the liquid transport element 222. Thus, the liquid transport element may be configured to transport liquid from the reservoir substrate to the heating element 224 (e.g., via capillary action). Representative types of substrates, reservoirs or other components for supporting the aerosol precursor composition are described in U.S. Pat. No. 8,528,569 to Newton and U.S. Pat. No. 8,715,070 to Davis et al.; U.S. Pat. App. Pub. No. 2014/0261487 to Chapman et al.; and U.S. patent application Ser. No. 14/170,838 to Bless et al., filed Feb. 3, 2014; which are incorporated herein by reference in their entireties.

As illustrated, the liquid transport element 222 may be configured to be in direct contact with the heating element 224. Various wicking materials, and the configuration and operation of those wicking materials within certain types of aerosol delivery devices, are set forth in U.S. Pat. No. 8,910,640 to Sears et al., which is incorporated herein by reference in its entirety. A variety of the materials disclosed by the foregoing documents may be incorporated into the present devices in various example implementations, and all of the foregoing disclosures are incorporated herein by reference in their entireties.

The heating element 224 may include a wire defining a plurality of coils wound about the liquid transport element 222. In some examples, the heating element may be formed by winding the wire about the liquid transport element as described in U.S. Pat. App. Pub. No. 2014/0157583 to Ward et al., which is incorporated herein by reference in its entirety. Further, in some examples, the wire may define a variable coil spacing, as described in U.S. Pat. App. Pub. No. 2014/0270730 to DePiano et al., which is incorporated herein by reference in its entirety. Various materials configured to produce heat when electrical current is applied therethrough may be employed to form the heating element. Example materials from which the wire coil may be formed include Kanthal (FeCrAl), Nichrome, Molybdenum disilicide (MoSi₂), molybdenum silicide (MoSi), Molybdenum disilicide doped with Aluminum (Mo(Si,Al)₂), graphite and graphite-based materials; and ceramic (e.g., a positive or negative temperature coefficient ceramic).

The first heating terminal 220a and the second heating terminal 220b (e.g., positive and negative terminals) at the opposing ends of the heating element 224 are configured to form an electrical connection with the control body 104 when the cartridge 102 is connected thereto. Further, when the control body is coupled to the cartridge, the control component 206 may form an electrical connection with the control body through the control component terminal. The control body may thus employ the control component to determine whether the cartridge is genuine and/or perform other functions. Examples of a suitable control component include one or more of each of a number of electronic components such as a microprocessor (individually or as part of a microcontroller), application-specific integrated circuit (ASIC), field-programmable gate array (FPGA) or the like. Further, various examples of control components and functions performed thereby are described in U.S. Pat. App. Pub. No. 2014/0096781 to Sears et al., which is incorporated herein by reference in its entirety.

Various other details with respect to the cartridge 102 are described above are provided in U.S. patent application Ser. No. 14/286,552 to Brinkley et al., filed May 23, 2014. Further, it should be understood that the cartridge may be assembled in a variety of manners and may include additional or fewer components which may be the same or different in other example implementations. For example, although the cartridge is generally described herein as including a reservoir substrate, in other examples, the cartridge may hold an aerosol precursor composition therein without the use of a reservoir substrate (e.g., through use of a container or vessel that stores the aerosol precursor composition or direct storage therein). In some examples, an aerosol precursor composition may be within a container or vessel that may also include a porous (e.g., fibrous) material therein. Further, in other examples, the aerosol precursor composition may be delivered to the atomizer via other mechanisms such as positive displacement mechanisms as disclosed in U.S. patent application Ser. No. 14/309,282, filed Jun. 19, 2014, bubble jet heads as disclosed in U.S. patent application Ser. No. 14/524,778, filed Oct. 29, 2014, and pressurized dispensers as disclosed in U.S. patent application Ser. No. 14/289,101, filed May 28, 2014, each to Brammer et al., each of which is incorporated herein by reference in its entirety. Additionally, although usage of a coil heating element is generally discussed herein, in other examples, the atomizer may include a microheater, one or more vaporization heating elements, and/or various atomizers as disclosed, for example, in U.S. patent application Ser. No. 14/309,282, filed Jun. 19, 2014; U.S. patent application Ser. No. 14/524,778, filed Oct. 29, 2014; and U.S. patent application Ser. No. 14/289,101, filed May 28, 2014, each to Brammer et al. and U.S. Pat. No. 8,881,737 to Collett et al., each of which is incorporated herein by reference in its entirety.

Various components of an aerosol delivery device according to the present disclosure can be chosen from components described in the art and commercially available. Reference is made for example to the reservoir and heater system for controllable delivery of multiple aerosolizable materials in an electronic smoking article disclosed in U.S. Pat. App. Pub. No. 2014/0000638 to Sebastian et al., which is incorporated herein by reference in its entirety.

Note further that portions of the cartridge 102 illustrated in FIG. 2 are optional. In this regard, by way of example, the cartridge may not include the flow director 208, the control component terminal 204, and/or the control component 206 in some example implementations.

In another example, substantially the entirety of the cartridge 102 may be formed from one or more carbon materials, which may provide advantages in terms of biodegradability and absence of wires. In this regard, the heating element may include carbon foam, the reservoir may include carbonized fabric, and graphite may be employed to form an electrical connection with the battery and controller. An example of a suitable carbon-based cartridge is provided in U.S. Pat. App. Pub. No. 2013/0255702 to Griffith et al., which is incorporated herein by reference in its entirety.

FIG. 3 illustrates an exploded view of the control body 104 of the aerosol delivery device 100 according to an example implementation of the present disclosure. As illustrated, in addition to the emissive or electronic paper display 106, the control body may include a coupler 302, an outer body 304, a sealing member 306, an adhesive member 308 (e.g., KAPTON® tape), a flow sensor 310 (e.g., a puff sensor or a pressure switch or sensor configured to detect a pressure drop or flow of air), a control component 312, a spacer 314, an electrical power source 316 (e.g., a battery, which may be rechargeable), a circuit board with an indicator 318 (e.g., a light emitting diode (LED)), a connector circuit 320, and an end cap 322. Examples of electrical power sources are described in U.S. Pat. App. Pub. No. 2010/0028766 to Peckerar et al., the disclosure of which is incorporated herein by reference in its entirety.

The aerosol delivery device 100 may incorporate a sensor or detector for control of supply of electric power to a heat generation element when aerosol generation is desired (e.g., upon draw during use). As such, for example, there is provided a manner or method for turning off the power supply to the heat generation element when the aerosol generating piece is not being drawn upon during use, and for turning on the power supply to actuate or trigger the generation of heat by the heat generation element during draw.

For example, with respect to the flow sensor 310, representative current regulating components and other current controlling components including various microcontrollers, sensors, and switches for aerosol delivery devices are described in U.S. Pat. No. 4,735,217 to Gerth et al.; U.S. Pat. No. 4,947,874 to Brooks et al.; U.S. Pat. No. 5,372,148 to McCafferty et al.; U.S. Pat. No. 6,040,560 to Fleischhauer et al.; U.S. Pat. No. 7,040,314 to Nguyen et al. U.S. Pat. No. 8,205,622 to Pan, and U.S. Pat. No. 8,881,737 to Collet et al.; U.S. Pat. Pub. Nos. 2009/0230117 to Fernando et al. and 2014/0270727 to Ampolini et al.; and U.S. patent application Ser. No. 14/209,191, filed Mar. 13, 2014, to Henry et al.; which are incorporated herein by reference in their entireties. Additional representative types of sensing or detection mechanisms, structures, components, configurations, and general methods of operation thereof, are described in U.S. Pat. No. 5,261,424 to Sprinkel, Jr.; U.S. Pat. No. 5,372,148 to McCafferty et al.; and PCT WO 2010/003480 to Flick; which are incorporated herein by reference in their entireties.

In one example, the indicator 318 may include one or more light emitting diodes. The indicator can be in communication with the control component 312 through the connector circuit 320 and illuminate, for example, during a user drawing on a cartridge (e.g., the cartridge 102) coupled to the coupler 302, as detected by the flow sensor 310. The end cap 322 may be adapted to make visible the illumination provided thereunder by the indicator. Accordingly, the indicator may illuminate during use of the aerosol delivery device 100 to simulate the lit end of a smoking article. However, in other examples, the indicator can be provided in varying numbers and can take on different shapes and can even be an opening in the outer body (such as for release of sound when such indicators are present).

Various elements that may be included in the control body are described in U.S. application Ser. No. 14/193,961 to Worm et al., filed Feb. 28, 2014, which is incorporated herein by reference in its entirety. Still further components can be utilized in the aerosol delivery device of the present disclosure. For example, U.S. Pat. No. 5,154,192 to Sprinkel et al. discloses indicators for smoking articles; U.S. Pat. No. 5,261,424 to Sprinkel, Jr. discloses piezoelectric sensors that can be associated with the mouth-end of a device to detect user lip activity associated with taking a draw and then trigger heating; U.S. Pat. No. 5,372,148 to McCafferty et al. discloses a puff sensor for controlling energy flow into a heating load array in response to a pressure drop through a mouthpiece; U.S. Pat. No. 5,967,148 to Harris et al. discloses receptacles in a smoking device that include an identifier that detects a non-uniformity in infrared transmissivity of an inserted component and a controller that executes a detection routine as the component is inserted into the receptacle; U.S. Pat. No. 6,040,560 to Fleischhauer et al. describes a defined executable power cycle with multiple differential phases; U.S. Pat. No. 5,934,289 to Watkins et al. discloses photonic-optronic components; U.S. Pat. No. 5,954,979 to Counts et al. discloses means for altering draw resistance through a smoking device; U.S. Pat. No. 6,803,545 to Blake et al. discloses specific battery configurations for use in smoking devices; U.S. Pat. No. 7,293,565 to Griffen et al. discloses various charging systems for use with smoking devices; U.S. Pat. No. 8,402,976 to Fernando et al. discloses computer interfacing means for smoking devices to facilitate charging and allow computer control of the device; U.S. Pat. No. 8,689,804 to Fernando et al. discloses identification systems for smoking devices; and WO 2010/003480 to Flick discloses a fluid flow sensing system indicative of a puff in an aerosol generating system; all of the foregoing disclosures being incorporated herein by reference in their entireties. Further examples of components related to electronic aerosol delivery articles and disclosing materials or components that may be used in the present article include U.S. Pat. No. 4,735,217 to Gerth et al.; U.S. Pat. No. 5,249,586 to Morgan et al.; U.S. Pat. No. 5,666,977 to Higgins et al.; U.S. Pat. No. 6,053,176 to Adams et al.; U.S. Pat. No. 6,164,287 to White; U.S. Pat. No. 6,196,218 to Voges; U.S. Pat. No. 6,810,883 to Felter et al.; U.S. Pat. No. 6,854,461 to Nichols; U.S. Pat. No. 7,832,410 to Hon; U.S. Pat. No. 7,513,253 to Kobayashi; U.S. Pat. No. 7,896,006 to Hamano; U.S. Pat. No. 6,772,756 to Shayan; U.S. Pat. Nos. 8,156,944 and 8,375,957 to Hon; U.S. Pat. No. 8,794,231 to Thorens et al.; U.S. Pat. No. 8,851,083 to Oglesby et al.; U.S. Pat. Nos. 8,915,254 and 8,925,555 to Monsees et al.; U.S. Pat. App. Pub. Nos. 2006/0196518 and 2009/0188490 to Hon; U.S. Pat. App. Pub. No. 2010/0024834 to Oglesby et al.; U.S. Pat. App. Pub. No. 2010/0307518 to Wang; U.S. Pat. App. Pub. No. 2014/0261408 to DePiano et al.; WO 2010/091593 to Hon; and WO 2013/089551 to Foo, each of which is incorporated herein by reference in its entirety.

Referring to FIGS. 1 and 2, during use, a user may draw on the mouthpiece 216 of the cartridge 102 of the aerosol delivery device 100. This may pull air through an opening in the control body 104 or in the cartridge. For example, in one example, an opening may be defined between the coupler 302 and the outer body 304 of the control body, as described in U.S. Pat. App. Pub. No. 2014/0261408 to DePiano et al., which is incorporated herein by reference in its entirety. However, the flow of air may be received through other parts of the aerosol delivery device in other example implementations. As noted above, in some examples, the cartridge may include the flow director 208. The flow director may be configured to direct the flow of air received from the control body 104 to the heating element 224 of the atomizer 210.

A sensor in the aerosol delivery device 100 (e.g., the flow sensor 310 in the control body 104) may sense the puff. When the puff is sensed, the control body 104 may direct current to the heating element 224 through a circuit including the first heating terminal 220a and the second heating terminal 220b. Accordingly, the heating element may vaporize the aerosol precursor composition directed to an aerosolization zone from the reservoir substrate 212 by the liquid transport element 222. Thus, the mouthpiece 216 may allow passage of air and entrained vapor (i.e., the components of the aerosol precursor composition in an inhalable form) from the cartridge 102 to a consumer drawing thereon.

As noted above, in some examples, the control body 104 may include the indicator 318 (e.g., an LED), which may be configured to illuminate an end of the control body. For example, the indicator may illuminate the end cap 322 during use of the aerosol delivery device 100 to simulate the lit end of a smoking article. However, it may be desirable to illuminate other or additional portions of an aerosol delivery device. Further, it may be desirable to transmit light within the aerosol delivery device to one or more locations positioned distally from the light source such that the position of the light source may be selected to facilitate assembly of the control body and/or provide other advantages.

Again, the emissive or electronic paper display 106 of example implementations of the present disclosure may be controllable to present information of the aerosol delivery device 100. This information may indicate, for example, an amount of aerosol precursor composition contained in or consumed from the cartridge 102 (e.g., reservoir substrate 212), or an amount of charge contained in or consumed from the power source 316. In some examples, this information may be presented in the form of icons.

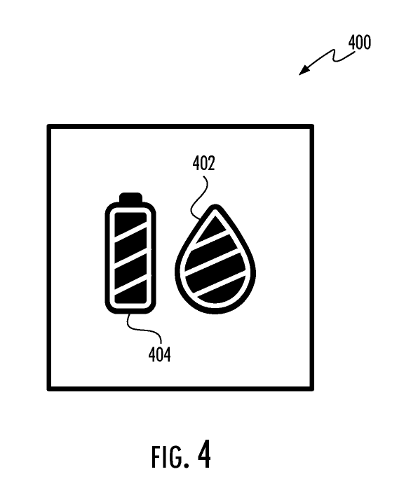

FIG. 4 illustrates information that may be presented by the emissive or electronic paper display 106, according to an example implementation of the present disclosure. As shown, the emissive or electronic paper display may present icons 402, 404 that represent respectively the aerosol precursor composition and power source 316. The icon for the aerosol precursor composition may indicate the amount thereof contained in or consumed from the cartridge 102 (e.g., reservoir substrate 212). Similarly, the icon for the power source may indicate an amount of charge contained in or consumed from the power source. As shown, each icon may be divided into a number of segments that represent incremental percentages (e.g., 10-20%) of its respective consumable. The icon for the aerosol precursor composition may be divided into segments that represent an incremental amount of remaining aerosol precursor composition (contained in the cartridge). And the icon for the power source may be divided into segments that represent an incremental amount of remaining charge (contained in the power source).

The foregoing description of use of the article(s) can be applied to the various example implementations described herein through minor modifications, which can be apparent to the person of skill in the art in light of the further disclosure provided herein. The above description of use, however, is not intended to limit the use of the article but is provided to comply with all necessary requirements of disclosure of the present disclosure. Any of the elements shown in the article(s) illustrated in FIGS. 1-4 or as otherwise described above may be included in an aerosol delivery device according to the present disclosure.

Many modifications and other implementations of the disclosure set forth herein will come to mind to one skilled in the art to which this disclosure pertains having the benefit of the teachings presented in the foregoing descriptions and the associated drawings. Therefore, it is to be understood that the disclosure is not to be limited to the specific implementations disclosed, and that modifications and other implementations are intended to be included within the scope of the appended claims. Moreover, although the foregoing descriptions and the associated drawings describe example implementations in the context of certain example combinations of elements and/or functions, it should be appreciated that different combinations of elements and/or functions may be provided by alternative implementations without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. An apparatus comprising:
at least one housing equipped with a heating element and containing an aerosol precursor composition, the heating element being configured to activate and vaporize components of the aerosol precursor composition; and
a flexible, emissive or electronic paper display on and contoured to the at least one housing, and controllable to present information of the aerosol delivery device,
wherein the apparatus is an aerosol delivery device or a cartridge for an aerosol delivery device.

2. The apparatus of claim 1, wherein the emissive or electronic paper display is contoured to a non-planar surface of the at least one housing.

3. The apparatus of claim 2, wherein the non-planar surface is substantially tubular in shape.

4. The apparatus of claim 1, wherein the emissive or electronic paper display is controllable to present information that indicates an amount of aerosol precursor composition contained in or consumed from the at least one housing.

5. The apparatus of claim 4, wherein the emissive or electronic paper display being controllable to present information includes being controllable to present an icon that represents the aerosol precursor composition and indicates the amount thereof contained in or consumed from the at least one housing.

6. The apparatus of claim 1, wherein the heating element is connected or connectable with a power source, and the emissive or electronic paper display is controllable to present information that indicates an amount of charge contained in or consumed from the power source.

7. The apparatus of claim 6, wherein the emissive or electronic paper display being controllable to present information includes being controllable to present an icon that represents the power source and indicates the amount of charge contained in or consumed therefrom.

8. The apparatus of claim 6, wherein the emissive or electronic paper display being controllable to present information includes being controllable to present information that also indicates an amount of aerosol precursor composition contained in or consumed from the at least one housing.

9. The apparatus of claim 8, wherein the emissive or electronic paper display being controllable to present information includes being controllable to present an icon that represents the power source and indicates the amount of charge contained in or consumed therefrom, and an icon that represents the aerosol precursor composition and indicates the amount thereof contained in or consumed from the at least one housing.

10. A control body coupled or coupleable with a cartridge that is equipped with a heating element and contains an aerosol precursor composition, the control body being coupled or coupleable with the cartridge to form an aerosol delivery device in which the heating element is configured to activate and vaporize components of the aerosol precursor composition, the control body comprising:
at least one housing;
a flexible, emissive or electronic paper display on and contoured to the at least one housing; and
a control component contained within the at least one housing and configured to operate in an active mode in which the control body is coupled with the cartridge, the control component in the active mode being configured to control the heating element to activate and vaporize components of the aerosol precursor composition, the control component being further configured to control the emissive or electronic paper display to present information of the cartridge, control body or aerosol delivery device.

11. The control body of claim 10, wherein the emissive or electronic paper display is contoured to a non-planar surface of the at least one housing.

12. The control body of claim 11, wherein the non-planar surface is substantially tubular in shape.

13. The control body of claim 10, wherein the control component is configured to control the emissive or electronic paper display to present information that indicates an amount of aerosol precursor composition contained in or consumed from the at least one housing.

14. The control body of claim 13, wherein the control component being configured to control the emissive or electronic paper display includes being configured to control the emissive or electronic paper display to present an icon that represents the aerosol precursor composition and indicates the amount thereof contained in or consumed from the at least one housing.

15. The control body of claim 10, wherein the heating element is connected or connectable with a power source, and the control component is configured to control the emissive or electronic paper display to present information that indicates an amount of charge contained in or consumed from the power source.

16. The control body of claim 15, wherein the control component being configured to control the emissive or electronic paper display includes being configured to control the emissive or electronic paper display to present an icon that represents the power source and indicates the amount of charge contained in or consumed therefrom.

17. The control body of claim 15, wherein the control component being configured to control the emissive or electronic paper display includes being configured to control the emissive or electronic paper display to present information that also indicates an amount of aerosol precursor composition contained in or consumed from the at least one housing.

18. The control body of claim 17, wherein the control component being configured to control the emissive or electronic paper display includes being configured to control the emissive or electronic paper display to present an icon that represents the power source and indicates the amount of charge contained in or consumed therefrom, and an icon that represents the aerosol precursor composition and indicates the amount thereof contained in or consumed from the at least one housing.
